(19) United States
(12) Patent Application Publication
(10) Pub. No.: US 2017/0291016 A1
(43) Pub. Date: Oct. 12, 2017

(54) MEDICAL PRESSURE GAUGE

(71) Applicant: Hideo FUMIYAMA, (US)

(72) Inventor: Hideo FUMIYAMA, Tokyo (JP)

(21) Appl. No.: 15/520,642

(22) PCT Filed: Oct. 20, 2015
(86) PCT No.: PCT/JP2015/079540

§ 371 Date: Apr. 20, 2017

(30) Foreign Application Priority Data
Oct. 22, 2014 (JP) 2014-215119

(51) Int. Cl. A61M 25/10; A61B 5/03

(52) U.S. Cl. CPC A61M 25/10184; A61M 25/10187; A61B 5/036; A61M 25/1018; A61B 5/4233

(57) ABSTRACT

Provided is a pressure gauge capable of being effectively used for internal pressure measurement of a balloon catheter and capable of providing easy monitoring of a pressure value even when measuring internal pressure of an extracorporeal circulation circuit. The pressure gauge measures internal pressure of a balloon catheter used for treatment and diagnosis or pressure of a body lumen of a living body and includes a coil-shaped tube 2 to elastically deform. One end of the tube 2 is set as an open fixed end and the other end is set as a sealed free end and pressure introduced from the open fixed end can be measured based on a displacement position of the tube 2 due to the pressure.

### TECHNICAL FIELD

The present invention relates to a medical pressure gauge to be used for medical treatment and diagnosis in the medical field.

### Background Art

A medical pressure gauge has been used for pressure measurement in various scenes of medical treatment such as measurement of internal pressure of a balloon catheter used for treatment and diagnosis, internal pressure of an extracorporeal circulation circuit which circulates body fluid such as blood outside a body, internal pressure of a blood vessel for sticking an injection needle, internal pressure of a trachea tube, and internal pressure of an abdominal cavity for treatment and diagnosis using an endoscope.

When treatment or diagnosis is performed using a balloon catheter, there is a case that measurement of internal pressure of a balloon is necessary for adjustment of pressure of the balloon catheter. In such a case, a capacity of a balloon is small in a balloon catheter for treatment of a coronary artery and a peripheral blood vessel having small diameter, or an intracorporeal lumen having small diameter such as a bile duct. Here the balloon is pressurized and expanded using an inflation device being a pressurizer and internal pressure of the balloon is measured with a pressure gauge built in the inflation device.

There have been two types available as a pressure gauge to be adopted for the inflation device; a Bourdon tube gauge and a digital pressure gauge which performs digital display using a semiconductor. Here, the Bourdon tube gauge includes a deformation member which elastically deforms in accordance with introduced pressure and displays the pressure with a scale indicated by an indicator rotating in accordance with displacement of the deformation member (e.g., see Patent Literature 1).

A balloon for high pressure is formed of a material such as nyron series, polyethylene terephthalate (PET), nylon thermoplastic elastomer (PEBAX), and polyethylene (PE) being semi-compliance or non-compliance with proof pressure of about atmospheric pressure used for a coronary artery or a peripheral artery. On the other hand, a balloon for low pressure has large compliance with material such as polyurethane, silicone, and latex and variation of internal pressure of the balloon is extremely small.

Accordingly, with the Bourdon tube gauge, since pressure is measured with extremely small variation, an operator is required to gaze the gauge. Therefore, the Bourdon tube gauge is inadequate for a pressure gauge to be used for a balloon catheter for aortic obstruction, a balloon catheter for cardiac valve treatment, a balloon used for transcatheter aortic valve treatment device or aortic aneurysm stent-graft inserting treatment, and further, a balloon catheter for treatment of an intracorporeal lumen with large diameter such as a balloon for an intracorporeal lumen used for trachea, esophagus and the like. Even when a digital pressure gauge is used, since a pressure value is required to be read with the first decimal point, reading is time consuming similarly to a case with a Bourdon tube gauge.

In contrast, a balloon catheter for aortic obstruction, a balloon catheter for cardiac valve treatment, and a balloon used for transcatheter aortic valve treatment device or aortic aneurysm stent-graft inserting treatment used for treatment of blood vessel with large diameter, and further, a balloon for intracorporeal lumen used for trachea or esophagus have relatively large capacity. Accordingly, since the capacity is insufficient with an inflation device, pressure is generally adjusted by pressurizing a balloon with a general purpose syringe having capacity of about 60 ml without using an inflation device in normal cases. However, such a syringe does not incorporate a pressure gauge and pressure cannot be measured thereby. Accordingly, when pressure is required to be measured, it has been required to use a plurality of the inflation devices described above having small capacity with a built-in pressure gauge. In such a case, it is required for a plurality of operators to operate the respective inflation devices to adjust expansion of the balloons while performing addition with the pressure gauges built in the respective inflation devices.

Further, in an artificial heart-lung circuit for surgical operation of cardiovascular, pressure measurement is performed for the purpose of monitoring pressure in the circuit. A centrifugal pump or a closed circuit is frequently used for the purpose of improving biocompatibility in the artificial heart-lung circuit in this case. Recently, a vacuum-assisted venous return method in which a blood storage tank is kept at negative pressure and a centrifugal pump blood removal method appropriately using venous bubble trap are becoming popular in view of advantages of achieving bloodless extracorporeal circulation by reducing a filling amount and enhancing minimally invasiveness owing to diameter thinning of a blood removing circuit and shortening of the circuit and in view of ensuring wide visual field at the time of intracardiac operation.

FIG. 14 illustrates a schematic structure of an extracorporeal circulation circuit 100 for vacuum-assisted venous return in an artificial heart-lung circuit 100. A main circuit 101 is configured of a reservoir 107 connected to a blood removal tube 114 returning from a patient, a centrifugal pump 108, an artificial lung 110, an artery filter 111, a blood transmission cannula 115 for transmitting to the patient, and a vent suction circuit 102 including a suction pump which collects and returns bleeding blood into the body and a vent pump which sucks excessive blood in the heart. To maintain the inside of the reservoir 107 at negative pressure, a negative pressure controller or a water trap 106 is connected, and further, a selective cerebral perfusion circuit 103, a myocardial protection circuit 104, a hemoconcentration circuit 105, or the like is connected corresponding to a case.

As an artificial lung 110, a membrane type artificial lung is used instead of a conventional bubble oxygenator which has not been used in view of biocompatibility and the like. Since a membrane of a membrane type artificial lung is made of porous polypropylene film, an accident that air is mixed into the blood circuit via a membrane of the membrane type artificial lung occurs when the blood side turns into negative pressure. At this time, internal pressure of the circuit at a blood removal side with a vacuum-assisted venous return method or a centrifugal pump blood removal method is negative pressure and an outlet side of the centrifugal pump is positive pressure. However, when a circuit using a roller pump such as the selective cerebral perfusion circuit 103 and the myocardial protection circuit 104 is connected to the outlet side of the centrifugal pump 108, such branch circuits suck up blood flow from the main circuit 101. In such a case, there is a risk that a bubble is mixed into the blood circuit owing to that positive pressure cannot be maintained at the blood side of the artificial lung 110 when a flow rate of the total branch circuits exceeds a flow rate of the centrifugal pump 108.

Further, in cases that blood removal flow rate cannot be secured and that blood storage level suddenly falls temporarily when hemorrhaging occurs, negative pressure increases. In such a case, flow rate of the branch circuits with roller pumps becomes larger than the capacity of the centrifugal pump when rotation frequency of the centrifugal pump is insufficient, so that internal pressure of the circuit at the outlet side of the centrifugal pump 108 turns to negative pressure and a risk that a bubble is mixed into the blood circuit increases. At this time, since a blood flowmeter which displays blood flow rate at the outlet side of the centrifugal pump 108 does not sound a warning alarm if the blood flow rate is larger than a lower limit of alarm setting of the flowmeter, an operator of the artificial heart-lung is required to pay close attention to internal pressure of the circuit from the outlet side of the centrifugal pump 108 to an inlet side of the artificial lung 110 not to overlook bubble mixing into the blood circuit via the membrane of the artificial lung.

Although a bubble detector which warns danger after a bubble is mixed is arranged at the artificial heart-lung circuit, when once a bubble is generated, any bubble in the artificial heart-lung circuit, artificial lung, arterial filter, and the like has to be removed with the artificial heart-lung device stopped. During the bubble removing operation, blood cannot be sent to a patient and the patient is caused to be in danger. Therefore, while monitoring internal pressure of the circuit and paying attention to the blood storage amount to prevent bubble mixing occurrence, it is required to comprehensively evaluate measured values of other various measuring apparatuses and to manage appropriately in a short period of time, for example, in 24 seconds in a worst case when a blood storage amount is two litters and a total transmission flow rate is 5 litters/minute. Accordingly, as a bubble mixing prevention measure, a pressure gauge being a Bourdon tube gauge to monitor internal pressure of the circuit is used.

However, since a gauge needle of the Bourdon tube gauge repeats action and vibration due to pressure variation caused by a roller pump of a branch circuit, the pressure gauge is required to be gazed continuously. Further, even when a digital pressure gauge with a semiconductor pressure sensor is used, since a displayed value is not changed for a prescribed time considering time for a person to read the value, pressure variation in short time during the prescribed time is not followed or an average value is displayed. Thus, there has been a problem that an accurate value cannot be comprehended.

### CITED LITERATURE

### Patent Literature

Patent Document 1: Japanese Patent Application Laid-Open No. 2009-31161

### SUMMARY OF THE INVENTION

Thus, a pressure gauge has not been used for internal pressure measurement of a balloon catheter used for treatment and diagnosis. Further, there has been a problem for actual use of a Bourdon tube gauge used when measuring internal pressure of an extracorporeal circulation circuit which circulates body fluid outside a body.

In view of the above, the present invention provides a pressure gauge capable of being effectively used for internal pressure measurement of a balloon catheter and capable of providing easy monitoring of a pressure value even when measuring internal pressure of an extracorporeal circulation circuit.

Accordingly, the present invention provides a medical pressure gauge to measure internal pressure of a balloon catheter used for treatment and diagnosis or pressure of a body lumen of a living body, including a cylindrical deforming member formed into a coil shape to elastically deform, wherein one end of the deforming member is set as an open fixed end and the other end is set as a sealed free end, and a displacement position of the sealed free end is configured to be capable of indicating pressure introduced from the open fixed end.

Here, the deforming member is a tube formed into a coil shape through thermal processing after being flattened through drawing processing.

The deforming member may be formed by inserting a coil-shaped core member to the inside of the flattened tube or, conversely, may be formed by inserting a coil member to a tube formed coil shape through thermal processing. Alternatively, a core member may not be inserted to the tube but bonded to a surface of the tube.

Meanwhile, a marker is arranged along a longitudinal direction on the surface of the deforming member to enable pressure introduced from the open fixed end to be measured based on a positional relation between the sealed free end and the marker in association with variation of a curvature radius of the coil shape caused by the pressure.

Even without arranging a marker, being used for an artificial heart-lung circuit, an atmospheric opened state, positive pressure state, and negative pressure state may be discriminated from a state of displacement of the sealed free end.

According to the present invention, a coil-shaped deforming member to elastically deform is included with one end of the deforming member being set as an open fixed end and the other end being set as a sealed free end. Here, pressure can be measured with displacement of a position of the sealed free end when the pressure is introduced from the open fixed end. Therefore, viewing angel is wide compared to a pressure gauge and measurement of pressure is facilitated.

### BRIEF DESCRIPTION OF THE DRAWINGS

FIGS. 1A to 1C illustrate a medical pressure gauge according to an embodiment of the present invention where FIG. 1A is a plane view, FIG. 1B is a side view, and FIG. 1C is a sectional view at A-A.

FIGS. 2A to 2C illustrate the medical pressure gauge according to the embodiment of the present invention when being pressurized where FIG. 2A is a plane view, FIG. 2B is a side view, and FIG. 2C is a sectional view at B-B.

FIG. 3A is a side view of the medical pressure gauge of FIG. 1A and FIGS. 3B and 3C are sectional views at C-C where FIG. 3B illustrates a structure that a core member is inserted to a tube and FIG. 3C illustrates a structure that the core member is bonded to the tube.

FIG. 4A is a side view of the medical pressure gauge of FIG. 2A and FIGS. 4B and 4C are sectional views at D-D where FIG. 4B illustrates a structure that the core member is inserted to the tube and FIG. 4C illustrates a structure that the core member is bonded to the tube.

FIGS. 5A and 5B is a front view of the medical pressure gauge of FIG. 1A with a scale arranged for pressure measurement.

FIG. 6 is an explanatory view of an example in which the medical pressure gauge according to the present invention is applied to a balloon catheter having large diameter to be used for a blood vessel and a body lumen having large diameter.

FIGS. 7A to 7C are explanatory views discriminating measurement pressure with a deforming state of the tube of the medical pressure gauge according to the present invention.

FIG. 8 is an explanatory view illustrating an example in which the medical pressure gauge according to the present invention is connected to a blood filter in an artificial heart-lung circuit for extracorporeal circulation.

FIG. 9 is an explanatory view illustrating another example in which the medical pressure gauge according to the present invention is connected to an artificial heart-lung circuit in an artificial heart-lung circuit for extracorporeal circulation.

FIG. 10 is an explanatory view illustrating an example of measuring blood pressure using the medical pressure gauge according to the present invention.

FIGS. 11A and 11B are front views of the medical pressure gauge according to the present invention indicating a scale for measurement of internal pressure of a cuff of an endotracheal tube and pressure in an abdominal cavity or a thoracic cavity at a surgery and the like using an endoscope.

FIG. 12 is an explanatory view illustrating an example of measuring internal pressure of a cuff of an endotracheal tube using the medical pressure gauge according to the present invention.

FIG. 13 is an explanatory view illustrating an example of measuring pressure in an abdominal cavity or a thoracic cavity at a surgical operation and the like using the medical pressure gauge according to the present invention.

FIG. 14 is structural view of an extracorporeal circulation circuit for vacuum-assisted venous return in an artificial heart-lung circuit.

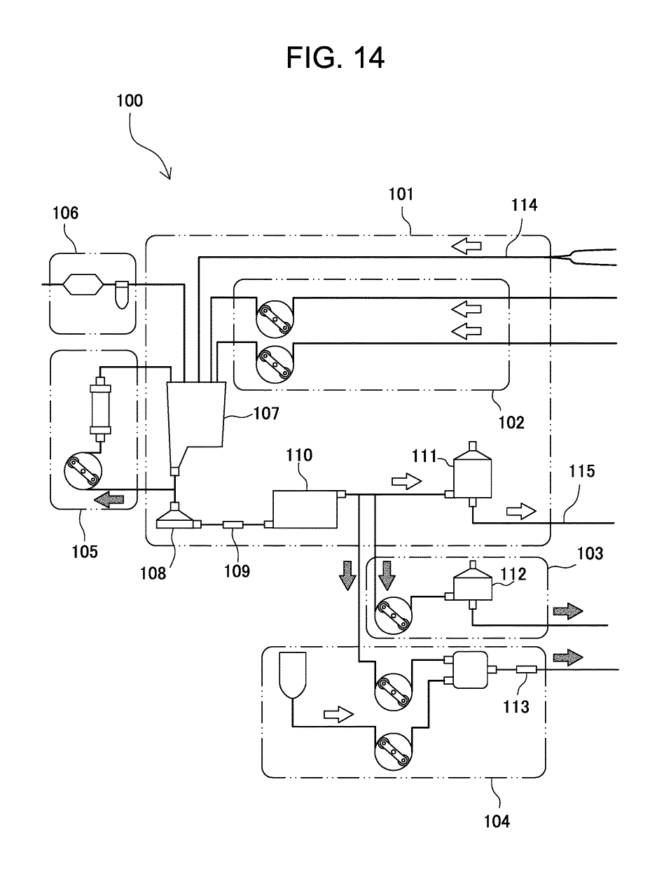

FIG. 15 is an explanatory view of a method to measure pressure in a general artery filter in a conventional artificial heart-lung circuit.

### EMBODIMENTS OF THE INVENTION

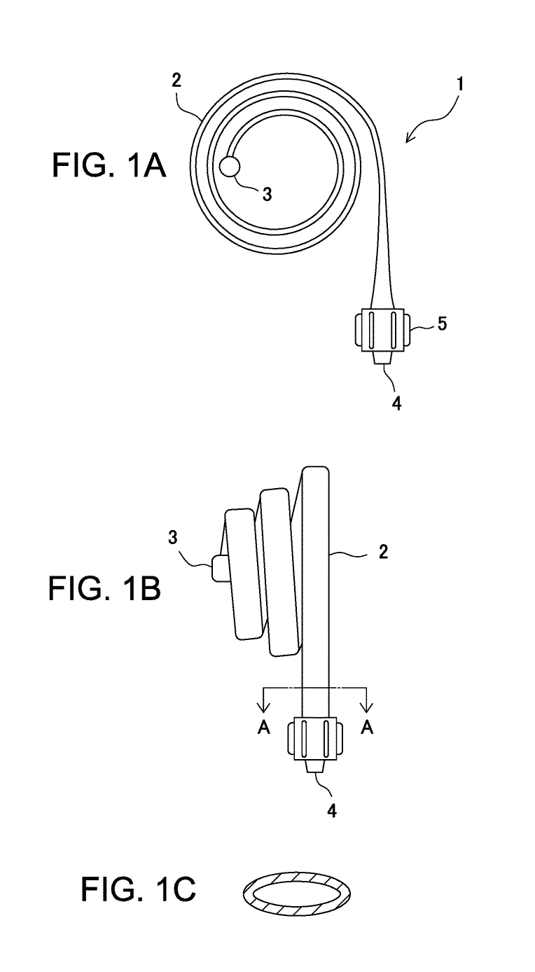

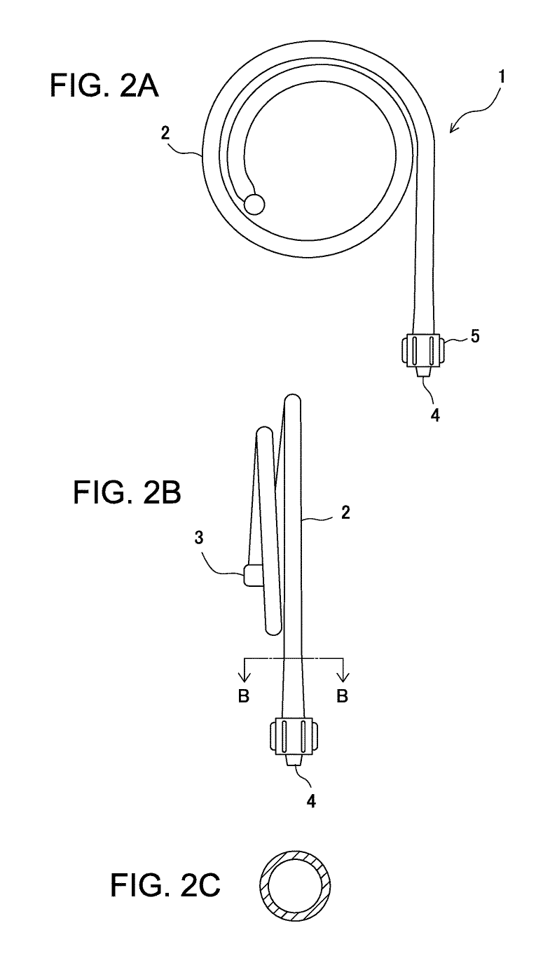

In the following, embodiments of the present invention will be described in detail with reference to the drawings. A medical pressure gauge 1 according to the present invention is structured of a deforming member wound in a coil shape as illustrated in FIGS. 1A to 2C.

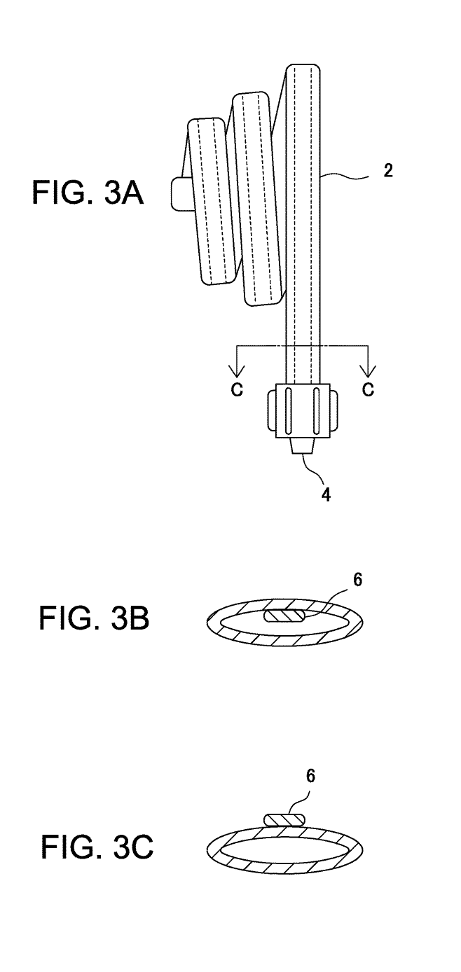

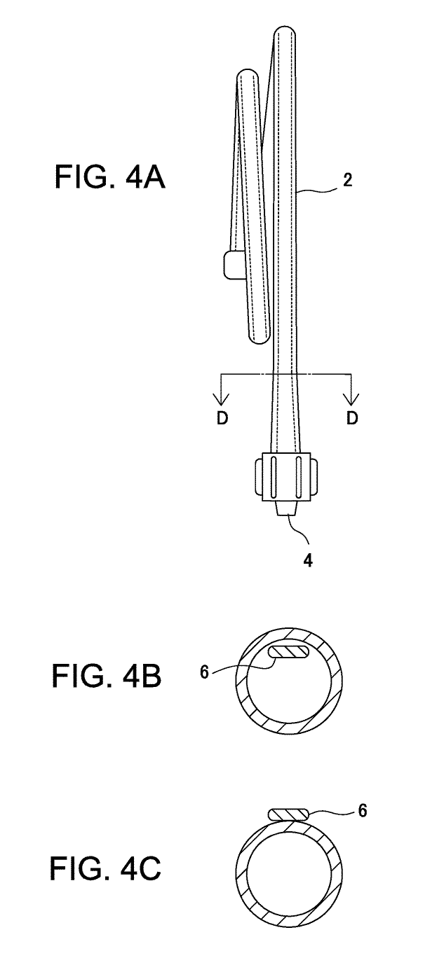

Instead of stainless such as SUS304, SUS310, and SUS316, at places where there is no contact with blood, alloy with high elasticity such as brass, aluminum brass, phosphor bronze, and beryllium copper formed into a coil shape are used as a core member 6 illustrated in FIGS. 3A to 4C. A base end of a tube 2 is an open fixed end 4 including a luer connector 5 to be connected to a measured portion and a leading end of the tube 2 is a sealed free end 3.

The tube 2 of the pressure gauge 1 illustrated in FIGS. 1A and 1B is formed into a coil shape through thermal processing after being flattened as illustrated in A-A section of FIG. 1C through drawing processing. On the other hand, the tube 2 of the pressure gauge 1 illustrated in FIGS. 2A and 2B illustrates that deformation occurs to increase the curvature radius of the coil shape as the section is deformed from a flat shape to a perfect circle when pressurized.

Molding of the tube 2 of the pressure gauge 1 illustrated in FIGS. 1A to 2C to the coil shape is performed by forming the tube 2 itself to the coil shape through thermal processing. However, molding may be performed with a core member 6 which is previously formed into a coil shape as illustrated in FIGS. 3A to 4C. The core member 6 in this case is formed of metal such as stainless or nitinol or synthetic resin and is formed into a coil shape through molding or thermal processing. The core member 6 is inserted to the tube 2 as illustrated in FIGS. 3B and 4B or the core member 6 is bonded to the surface of the tube 2 as illustrated in FIGS. 3C and 4C. Then, the tube 2 is formed into a coil shape.

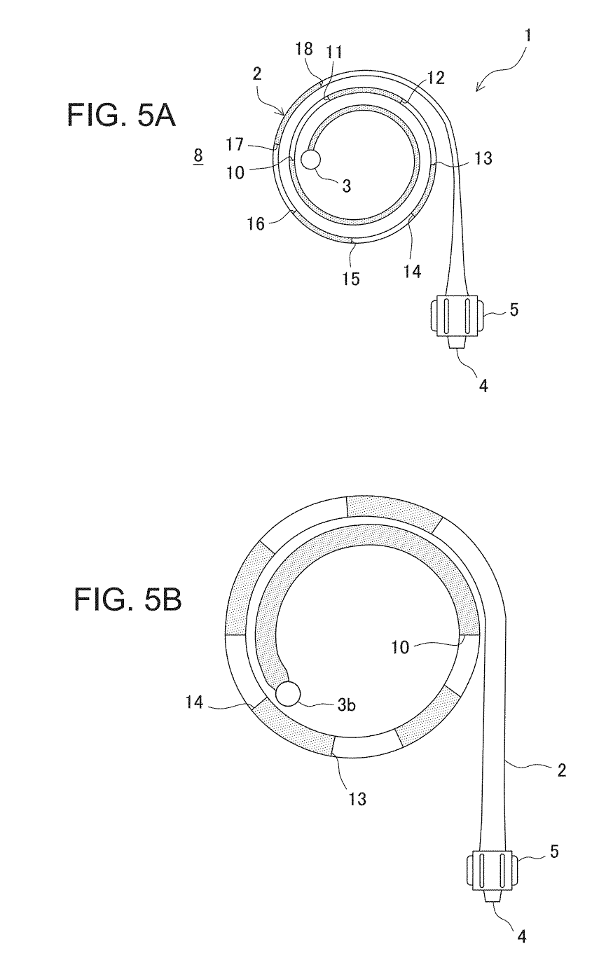

FIGS. 5A and 5B are front views of the pressure gauge 1 illustrated in FIG. 1A in with a scale for pressure measurement arranged, where FIG. 5A illustrates an atmospheric opened state before pressurizing and FIG. 5B illustrates a pressurized state.

Markers 8 are printed on a surface of the tube 2 of the pressure gauge 1 and pressure is indicated by a position where the sealed free end 3 is in contact with the marker. The illustrated markers 8 include nine pressure markers where a pressure marker 10 which is in contact with the sealed free end 3 in the atmospheric opened state indicates the pressure in the atmospheric opened state, pressure marker 11 indicates 0.1 ATM, pressure marker 12 indicates 0.2 ATM, pressure marker 13 indicates 0.3 ATM, pressure marker 14 indicates 0.4 ATM, pressure marker 15 indicates 0.5 ATM, pressure marker 16 indicates 0.6 ATM, pressure marker 17 indicates 0.7 ATM, and pressure marker 18 indicates 0.8 ATM.

FIG. 5B illustrates a pressurized state when measurement pressure is introduced from the open fixed end 4. Since the section of the tube 2 is expanded and the curvature radius of the winding is increased due to the introduction of the measurement pressure, the sealed free end 3 moves in a direction to unwind the winding. Owing to the above, the sealed free end 3 becomes apart from a position opposing to the pressure marker 10 and moves to a position between the pressure marker 13 and the pressure marker 14. Being near the pressure marker 14, it is indicated that the measurement pressure is higher than 0.35 ATM and lower than 0.4 ATM. Thus, when measurement pressure is introduced, pressure is measured by confirming the displaced position of the sealed free end 3 with respect to the pressure marker. It is preferable to print the respective pressure markers with different colors for visual observation.

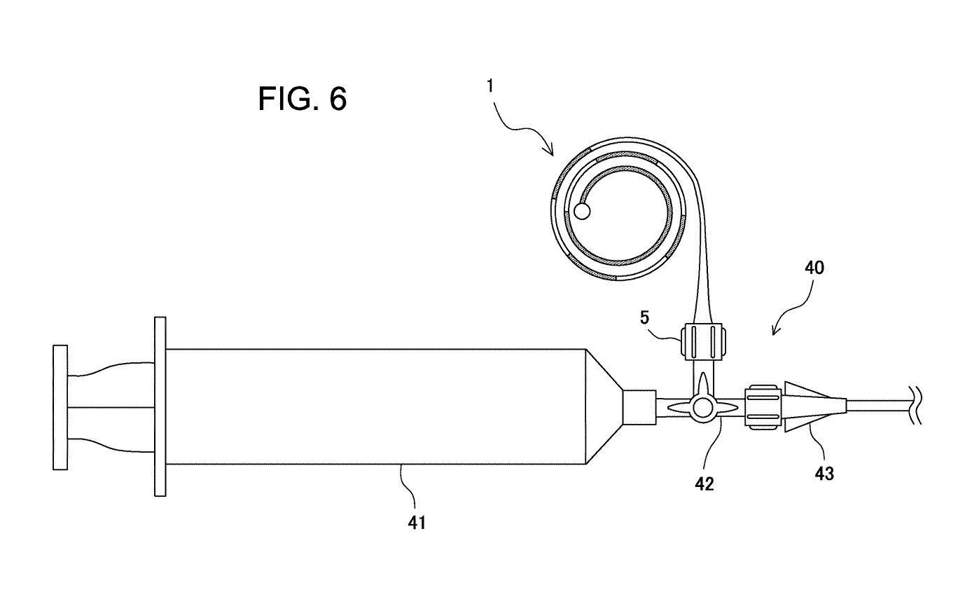

FIG. 6 illustrates an example in which the medical pressure gauge 1 according to the present invention is applied to a balloon catheter 40 having large diameter to be used for a blood vessel and a body lumen having large diameter. In this case, the balloon catheter 40 is connected to a syringe 41 with high versatility having capacity of about 60 ml via a three way stopcock 42, so that the balloon is pressurized with the syringe 41 to adjust pressure. At this time, by connecting the luer connector 5 of the pressure gauge 1 to one side of the three way stopcock 42, the sealed free end 13b indicates one of the pressure markers 11 to 18 in accordance with the pressure even with an extremely weak pressure being the balloon expanding internal pressure. Accordingly, the pressure can be easily confirmed with visual observation from a distanced place.

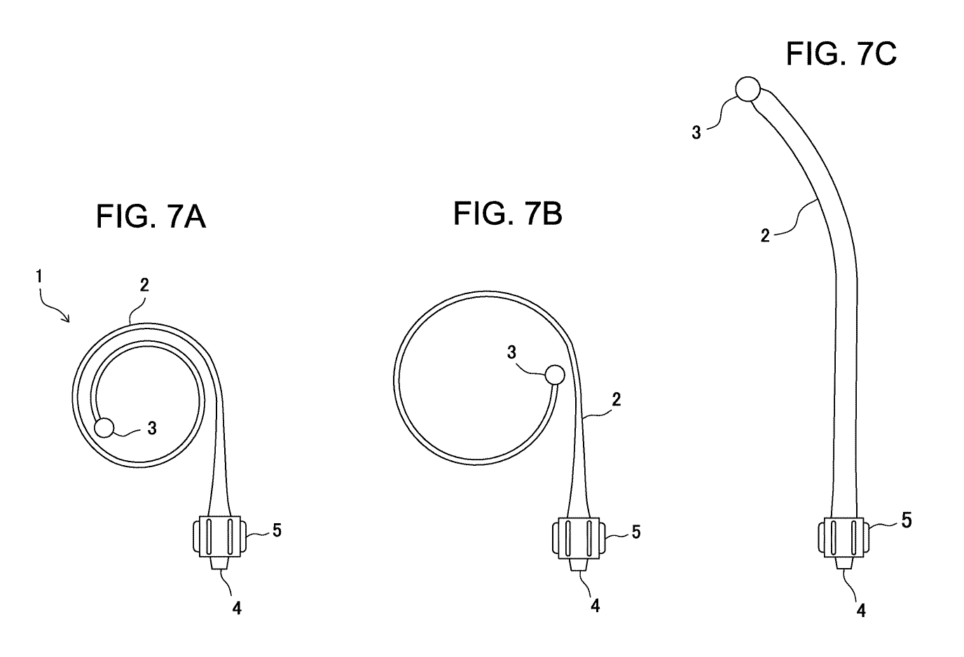

The pressure gauge 1 illustrated in FIGS. 7A to 7C enables an observer to easily discriminate measurement pressure even from a distanced place. Pressure markers are not arranged at the pressure gauge 1 illustrated in FIGS. 7A to 7C and pressure is discriminated with a deforming state of the tube 2 due to introduction of measurement pressure from the open fixed end 4. In an atmospheric opened state, the sealed free end 3 of the tube 2 is positioned to be singly wound as illustrated in FIG. 7B. When the pressure to measure is negative pressure, the sealed free end 3 is positioned to be doubly wound as illustrated in FIG. 7A. When the pressure to measure is positive pressure, the wound state of the sealed free end 3 is released and the tube 2 is to be in an approximately straight state as illustrated in FIG. 7C.

Accordingly, the pressure can be confirmed whether being in an atmospheric opened state, positive pressure, or negative pressure with the tube 2 being in a singly wound state, a doubly wound state, or a straight state. When the pressure gauge 1 is used to measure internal pressure of an artificial heart-lung circuit for extracorporeal circulation, it is preferable to set the tube 2 to be in an approximately straight state with the measurement pressure being about 300 mmHg. When the pressure gauge 1 is used to measure internal pressure of a blood vessel for sticking a syringe needle or the like, it is preferable to set the tube 2 to be in an approximately straight state with the measurement pressure being about 100 mmHg.

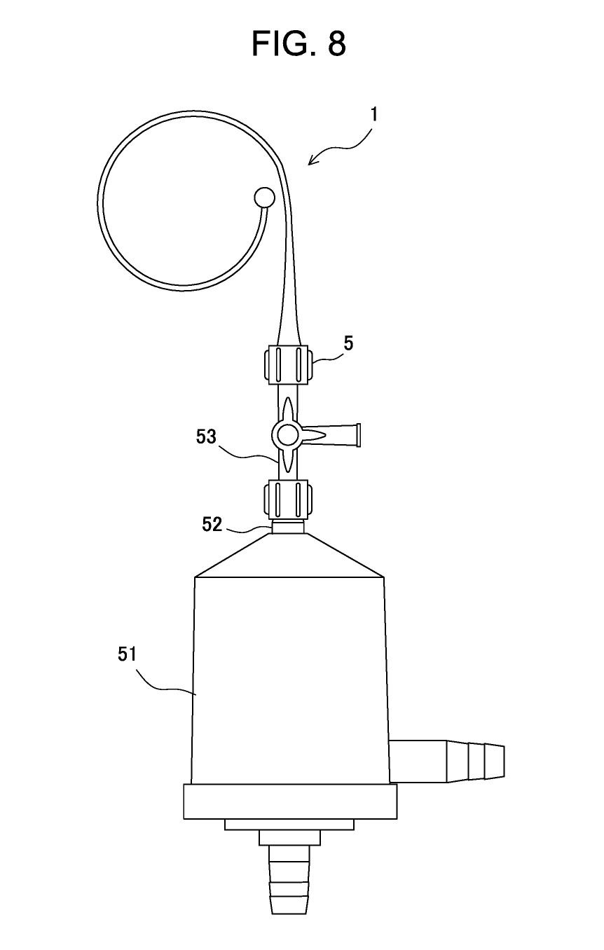

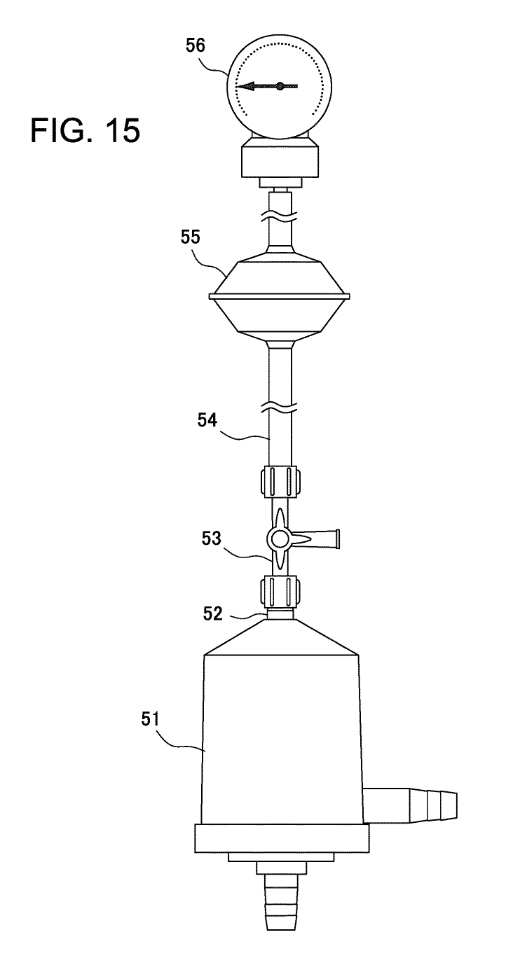

FIG. 8 illustrates an example in which the pressure gauge 1 illustrated in FIGS. 7A to 7C is connected to an artificial heart-lung circuit for extracorporeal circulation. A method to measure pressure in a general arterial filter in a conventional artificial heart-lung circuit will be described with reference to FIG. 15. A three way stopcock 53 is connected to a vent port 52 at an upper part of an arterial filter 51 and a Bourdon tube gauge 56 is connected thereto via an extension tube 54 for blood pressure measurement and a pressure separator 55. Since the Bourdon tube gauge 56 is a non-sterilization item to be reused, the extension tube 54 for blood pressure measurement and the pressure separator 55 are required to prevent contamination to the artificial heart-lung circuit, so that the device is increased in size and cost is increased.

However, as illustrated in FIG. 8, by directly connecting the pressure gauge 1 illustrated in FIGS. 7A to 7C to the vent port 52 at the upper part of the arterial filter 51 of the artificial heart-lung circuit with a luer connector via the three way stopcock 53, the extension tube 54 for blood pressure measurement and the pressure separator 55 become unnecessary. The pressure gauge 1 indicates that pressure at the inlet port side of the arterial filter 51 is in an atmospheric opened state when the tube 2 is singly wound, in a negative pressure state when the tube 2 is doubly wound, and in a positive pressure state when the tube 2 is in an approximately straight state.

Thus, the extension tube 54 for blood pressure measurement and the pressure separator 55 become unnecessary. Further, pressure in each of an atmospheric opened state, positive pressure state, and negative pressure state at the inlet port side of the arterial filter 51 can be easily recognized from a distanced place with the deforming state of the tube 2 corresponding to the measurement pressure.

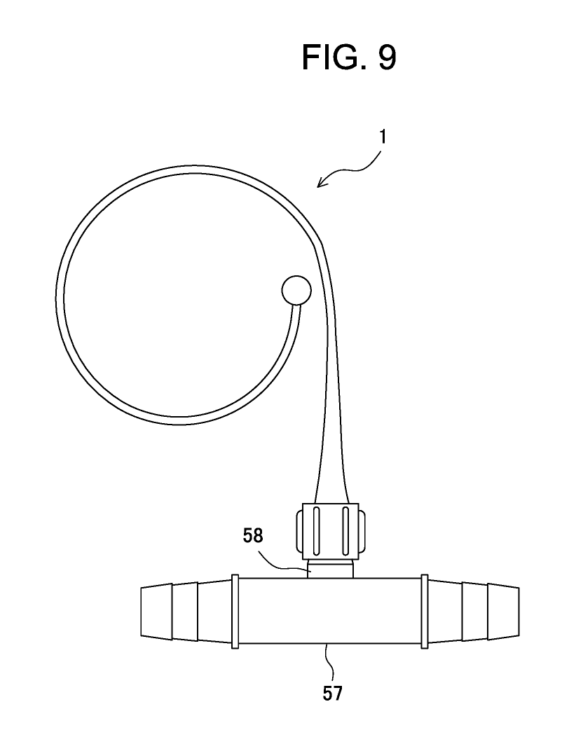

In FIG. 9, the pressure gauge 1 illustrated in FIGS. 7A to 7C is connected to a luer lock connector 58 at the upper part of an artificial heart-lung circuit connector 57 of the artificial heart-lung circuit for extracorporeal circulation. Here, pressure in the artificial heart-lung circuit in each of an atmospheric opened state, positive pressure state, and negative pressure state can be easily recognized from a distanced place with deforming states of the tube 1 corresponding to the pressure.

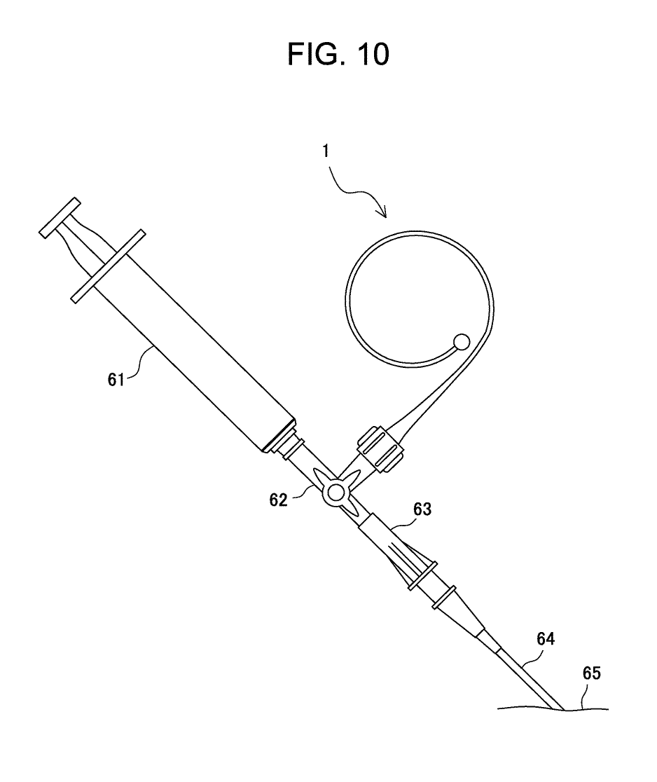

In FIG. 10, a three way stopcock 62 and an intradermal needle 63 and an external cylinder 64 of a puncture needle are connected to an injector 61. Owing to that the pressure gauge 1 illustrated in FIG. 8 is connected to the three way stopcock 62, when the intradermal needle 63 reaches a deep blood vessel via skin 65, blood pressure thereof is reflected to the pressure gauge 1, so that an artery and a vein of the deep blood vessel can be easily discriminated with the deforming state of the tube 1 in accordance with the blood pressure. Since blood pressure can be easily measured by connecting the pressure gauge 1 to the injector 61, an artery and a vein of a deep blood vessel can be accurately discriminated even for a patient of low output syndrome or the like by a non-skilled person.

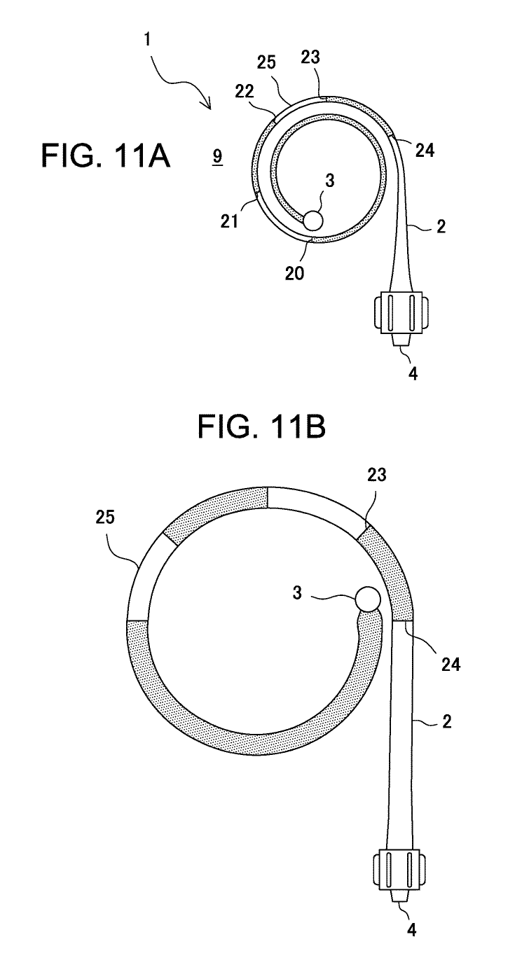

FIGS. 11A and 11B are front views of the pressure gauge 1 with a scale for measurement of internal pressure of a cuff of an endotracheal tube or pressure in an abdominal cavity or a thoracic cavity at a surgery and the like using an endoscope. A marker 9 including five pressure markers corresponding to measurement pressure in the abdominal cavity or the thoracic cavity is printed on the tube 2 of the pressure gauge 1. Pressure is indicated by a position where the sealed free end 3 is in contact with the pressure marker. Colors indicating risk in stages such as blue, yellow, and red following a traffic signal are preferable for the colors of the markers.

FIG. 11A illustrates an atmospheric opened state before pressurizing and FIG. 11B illustrates a pressurized state. In an atmospheric opened state, the sealed free end 3 is opposed to a pressure marker 20. Accordingly, the pressure marker 20 indicates the pressure in the atmospheric opened state. Further, pressure marker 21 indicates 10 mmHg, pressure marker 22 indicates 20 mmHg, pressure marker 23 indicates 30 mmHg, and pressure marker 24 indicates 40 mmHg. Accordingly, when the tube 2 of the pressure gauge 1 is located between the pressure marker 23 and the pressure marker 24, it is indicated that pressure is between 30 mmHG and 40 mmHg. Since the sealed free end 30 is located near the marker 24 in this case, it can be confirmed that the pressure is higher than 35 mmHg and lower than 40 mmHg.

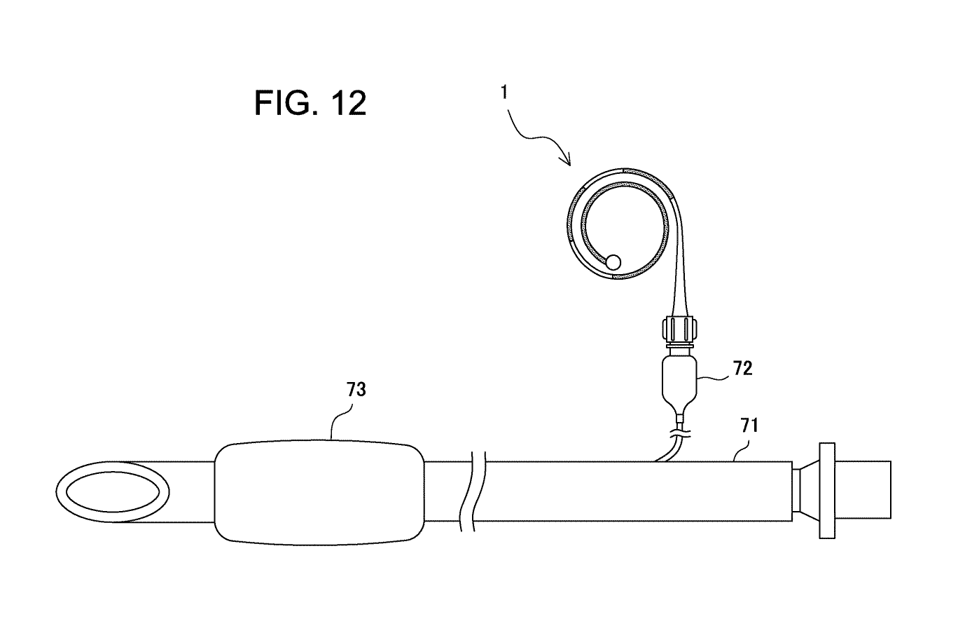

FIG. 12 illustrates an example of performing internal pressure measurement of a cuff 73 of an endotracheal tube 71 using the pressure gauge 1a of FIG. 5A.

In internal pressure measurement of an endotracheal tube and the like, when internal pressure of a cuff of an endotracheal tube, a tracheotomy tube, or the like is low, respiratory air of positive pressure ventilation leaks from a gap between the cuff and a tracheal wall to obstruct ventilation, the respiratory tract cannot be protected from misswallowing of oral discharge and reverse flow of gastric contents, and laughter gas leaks to an operating room in an operation with general anesthesia. On the other hand, when internal pressure of the cuff is high, bilane and necrosis of tracheal mucosa is caused due to blood circulation failure of tracheal wall tissue, resulting in defluxion of cilia and obstruction of spewing impurities in the lung to extrathoracic as a sputum in addition to complicating disease such as bleeding, tracheoesophageal fistula, and granulation.

To prevent the above, cuff pressure is required to be maintained at appropriate pressure of about 20 to 30 mmHg. However, in internal pressure measurement of a cuff, adjustment of a degree of expanding of a pilot balloon 72 connected to the cuff of the endotracheal tube 71 is performed with touch sense of a hand. When a cuff internal pressure gauge is used to measure accurate internal pressure, since connection is performed every time measurement is to be performed, attention has to be payed for handling. Here, there may be a method to connect the endortracheal tube 71 and the cuff internal pressure gauge with along tube for pressure measurement and to continuously perform monitoring. However, with this method, there is a risk that disconnection occurs due to movement of a patient and attention has to be constantly payed to connection while sufficiently considering routing of the tube for pressure measurement.

In the example illustrated in FIG. 12, the pressure gauge 1 is connected to the pilot balloon 72 of the endortracheal tube 71 so that internal pressure of the cuff 73 of the endortracheal tube 71 can be easily measured.

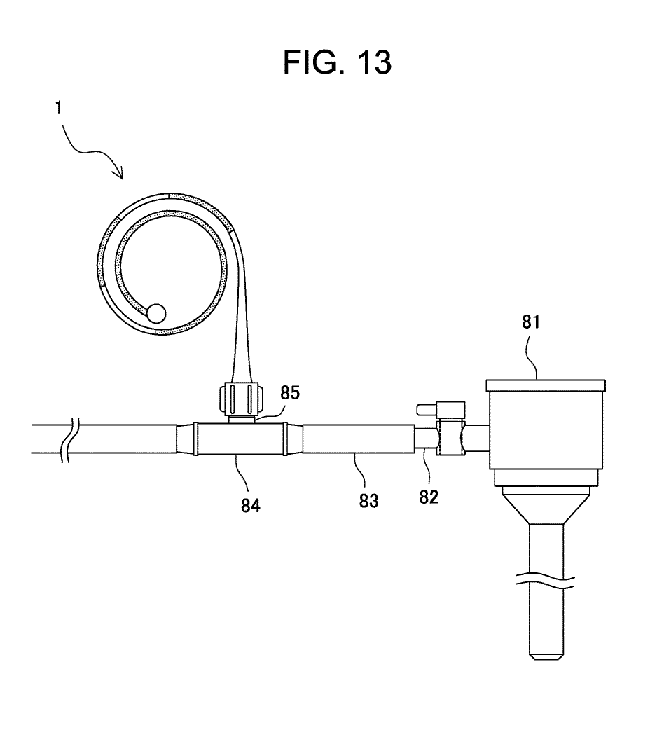

FIG. 13 illustrates an example of measuring pressure, using the pressure gauge 1 illustrated in FIG. 5A, in an abdominal cavity or a thoracic cavity at a surgical operation and the like using an endoscope or a soft or hard mirror.

In an endoscope surgical operation using a pneumoperitoneum device, operating field cannot be secured when pressure in an abdominal cavity or a thoracic cavity is low, and pressing and closing of a venous blood vessel is caused when pressure is high. To prevent the above, controlling pressure of a pneumoperitoneum device and the like is required. In this case, the pressure is required to be controlled to 20 mmHg or lower. To confirm the pressure, an operator has to have another manipulator to confirm or confirm by oneself while turning one's eye vision away from an endoscope at each time. Confirming is time-consuming in both cases.

In the example illustrated in FIG. 13, a tube connector 84 is connected to a tube 83 for pneumoperitoneum which is connected to a pneumoperitoneum port 82 of a trocar 81 at an operation and the like using an endoscope and the pressure gauge 1 is connected to the luer lock connector 85 at the upper part of the tube connector 84, so that pressure in an abdominal cavity or a thoracic cavity can be easily recognized with the displacement position of the sealed free end 3.

As described above, a medical pressure gauge according to the present invention can be used to measure internal pressure of a balloon catheter used for treatment and diagnosis, internal pressure of an extracorporeal circulation circuit which circulates body fluid such as blood outside a body, internal pressure of a blood vessel for puncturing an injection needle, internal pressure of a trachea tube and the like, and internal pressure of an abdominal cavity and the like for treatment and diagnosis using an endoscope. Accordingly, a measurer can easily recognize measurement result.

In the above, embodiments of the present invention are described. Here, the present invention is not limited to the embodiments described above, and various modifications can be applied based on the spirit of the present invention and such modifications are not excluded from the scope of the present invention. cl INDUSTRIAL APPLICABILITY

The present invention relates to a medical pressure gauge effectively used for measurement of internal pressure of a balloon catheter used for treatment and diagnosis and internal pressure of a body cavity and has industrial applicability.

### EXPLANATION OF REFERENCES

1 Medical pressure gauge
2 Tube (deforming member)
3 Sealed free end
4 Open fixed end
5 Luer connector
6 Core material
8 Marker
9 Marker

1. A medical pressure gauge to measure internal pressure of a balloon catheter used for treatment and diagnosis or pressure of a body lumen of a living body, comprising
a cylindrical deforming member formed into a coil shape to elastically deform,
wherein one end of the deforming member is set as an open fixed end and the other end is set as a sealed free end, and
a marker is arranged along a longitudinal direction on the surface of the deforming member to enable pressure introduced from the open fixed end to be measured based on a positional relation between the sealed free end and the marker in association with variation of a curvature radius of the coil shape caused by the pressure introduced to the open fixed end.

2. The medical pressure gauge according to claim 1,
wherein the deforming member is a tube formed into a coil shape through thermal processing after being flattened through drawing processing.

3. The medical pressure gauge according to claim 1,
wherein the deforming member is a tube formed by inserting a coil-shaped core member to the inside of the tube.

4. The medical pressure gauge according to claim 1,
wherein the deforming member is a tube formed by inserting a coil-shaped core member to the inside of a flattened tube.

5. The medical pressure gauge according to claim 1,
wherein the deforming member is formed by inserting a core member to a tube formed into a coil shape through thermal processing.

6. The medical pressure gauge according to claim 1,
wherein the deforming member is formed by bonding a core member to the outside of a tube.

7. (canceled)

8. The medical pressure gauge according to claim 1 to be used for an artificial heart-lung circuit, the medical pressure gauge being configured to discriminate an atmospheric opened state, positive pressure state, and negative pressure state from a state of displacement of the sealed free end.
